(19) United States
(12) Patent Application Publication
(10) Pub. No.: US 2025/0384977 A1
(43) Pub. Date: Dec. 18, 2025

(54) HEALTH DATA MANAGEMENT METHOD AND APPARATUS, ELECTRONIC DEVICE, AND READABLE STORAGE MEDIUM

(71) Applicant: BOE TECHNOLOGY GROUP CO., LTD., Beijing (CN)

(72) Inventors: Yubin Zhou, Beijing (CN); Guoqiang Zhang, Beijing (CN); Qingbing Liu, Beijing (CN); Yan Li, Beijing (CN); Endi Huang, Beijing (CN)

(73) Assignee: BOE TECHNOLOGY GROUP CO., LTD., Beijing (CN)

(21) Appl. No.: 18/877,360

(22) PCT Filed: May 10, 2023
(86) PCT No.: PCT/CN2023/093234

§ 371 Date: Dec. 20, 2024

(30) Foreign Application Priority Data
Jun. 22, 2022 (CN) 202210713607.9

(51) Int. Cl. G16H 10/60; G06F 21/31

(52) U.S. Cl. CPC G16H 10/60; G06F 21/31

(57) ABSTRACT

The present disclosure provides a health data management method, a health data management apparatus, an electronic device, and a readable storage medium. The health data management method is applied to a health management server in communication with an Internet-of-Things health detection terminal, and includes: receiving health detection data associated with a target user from the Internet-of-Things health detection terminal; establishing a biological model of the target user in accordance with the health detection data; and verifying the biological model, and generating a health detection result of the target user in accordance with the verified biological model.

### CROSS-REFERENCE TO RELATED APPLICATIONS

This application claims a priority of the Chinese Patent Application No. 202210713607.9 filed on Jun. 22, 2022, which is incorporated herein by reference in its entirety

### TECHNICAL FIELD

The present disclosure relates to the field of the Internet-of-Things technology, and in particular to a health data management method, a health data management apparatus, an electronic device and a readable storage medium.

### BACKGROUND

Along with the continuous improvement of people's awareness of health, people pay more and more attention to their own health monitoring, and the scope and indicators of health detection are increasing. With the development of information technology, especially the development of Internet-of-Things technology, the application of health detection scenario in accordance with open Internet-of-Things has become a trend, so as to facilitate collection and subsequent management of physical data.

### SUMMARY

An object of the present disclosure is to provide a health data management method, a health data management apparatus, an electronic device, and a readable storage medium, so as to solve the problem in the related art.

In one aspect, the present disclosure provides in some embodiments a health data management method applied to a health management server in communication with an Internet-of-Things health detection terminal, including: receiving health detection data associated with a target user from the Internet-of-Things health detection terminal; establishing a biological model of the target user in accordance with the health detection data; and verifying the biological model, and generating a health detection result of the target user in accordance with the verified biological model.

In a possible embodiment of the present disclosure, the biological model includes a plurality of detection sub-models corresponding to different detection items. The verifying the biological model includes: verifying a sub-model matching degree of each detection sub-model in accordance with reference data matching the target user; and generating a verification result of the biological model in accordance with the sub-model matching degree.

In a possible embodiment of the present disclosure, the verifying the sub-model matching degree of each detection sub-model in accordance with the reference data matching the target user includes: obtaining an association relationship among at least a part of the detection sub-models; and verifying the sub-model matching degree of each detection sub-model in accordance with the association relationship.

In a possible embodiment of the present disclosure, the detection sub-model includes one or more of a blood pressure sub-model, a blood glucose sub-model, a blood oxygen sub-model, a body fat sub-model, a body composition sub-model, a blood pressure sub-model, a bone substance sub-model, a lung function sub-model, an arteriosclerosis sub-model or an electrocardio sub-model.

In a possible embodiment of the present disclosure, prior to verifying a confidence level of the biological model, the method further includes: obtaining association information about the health detection data, the association information including at least one of an environmental factor or a physical factor of the target user; and generating a constraint condition for verifying the biological model in accordance with the association information.

In a possible embodiment of the present disclosure, the environmental factor includes one or more of a collection period, weather information or geographical environment; and/or the physical factor includes one or more of food-intake information or health information.

In a possible embodiment of the present disclosure, subsequent to receiving the health detection data associated with the target user from the Internet-of-Things health detection terminal, the method further includes: invoking a data filtering rule corresponding to the health detection data, the data filtering rule including one or more of a numerical range rule, a data collection state rule or a data format rule; and eliminating abnormal data in the health detection data in accordance with the data filtering rule.

In a possible embodiment of the present disclosure, prior to receiving the health detection data associated with the target user from the Internet-of-Things health detection terminal, the method further includes: receiving login information from the Internet-of-Things health detection terminal; verifying the login information in accordance with user information about the target user; and transmitting a verification result for the login information to the Internet-of-Things health detection terminal.

In another aspect, the present disclosure provides in some embodiments a health data management apparatus applied to a health management server in communication with an Internet-of-Things health detection terminal, including: a health detection data reception module configured to receive health detection data associated with a target user from the Internet-of-Things health detection terminal; a biological model establishment module configured to establish a biological model of the target user in accordance with the health detection data; and a verification module configured to verify the biological model, and generate a health detection result of the target user in accordance with the verified biological model.

In yet another aspect, the present disclosure provides in some embodiments an electronic device, including a memory, a processor, and a program stored in the memory and executed by the processor. The processor is configured to read the program in the memory so as to implement the steps of the above-mentioned health data management method.

In still yet another aspect, the present disclosure provides in some embodiments a readable storage medium storing therein a program. The program is executed by a processor, so as to implement the steps of the above-mentioned health data management method.

### BRIEF DESCRIPTION OF THE DRAWINGS

In order to illustrate the technical solutions of the present disclosure in a clearer manner, the drawings desired for the present disclosure will be described hereinafter briefly. Obviously, the following drawings merely relate to some embodiments of the present disclosure, and based on these drawings, a person skilled in the art may obtain the other drawings without any creative effort.

FIG. 1 is a flow chart of a health data management method according to one embodiment of the present disclosure;

FIG. 2 is another flow chart of the health data management method according to one embodiment of the present disclosure;

FIG. 3 is a schematic view showing a health data management apparatus according to one embodiment of the present disclosure; and

FIG. 4 is a block diagram of an electronic device according to one embodiment of the present disclosure.

### DETAILED DESCRIPTION

In order to make the objects, the technical solutions and the advantages of the present disclosure more apparent, the present disclosure will be described hereinafter in a clear and complete manner in conjunction with the drawings and embodiments. Obviously, the following embodiments merely relate to a part of, rather than all of, the embodiments of the present disclosure, and based on these embodiments, a person skilled in the art may, without any creative effort, obtain the other embodiments, which also fall within the scope of the present disclosure.

Such words as “first” and “second” involved in the embodiments of the present disclosure are merely used to differentiate different objects rather than to represent any specific order. In addition, such terms as “include” or “including” or any other variations involved in the present disclosure intend to provide non-exclusive coverage, so that a procedure, method, system, product or device including a series of steps or units may also include any other elements not listed herein, or may include any inherent steps or units of the procedure, method, system, product or device. In addition, the expression “and/or” in the embodiments of the present disclosure is merely used to represent at least one of the objects before and after the expression. For example, “A and/or B and/or C” represents seven situations, i.e., there is only A, there is only B, there is only C, there are both A and B, there are both B and C, thereby are both A and C, and there are A, B and C.

The present disclosure provides in some embodiments a health data management method.

In the embodiments of the present disclosure, the health data management method is applied to a health management server, and the health management server is in communication with an Internet-of-Things health detection terminal.

The Internet-of-Things health detection terminal is arranged in, but not limited to, a health room.

In the embodiments of the present disclosure, the health room refers to a health detection station which is open to be public to some extent and where various Internet-of-Things health detection terminals are provided. Physical detection is performed by a user using these Internet-of-Things health detection terminals so as to obtain detection data.

In some embodiments of the present disclosure, each Internet-of-Things health detection terminal is in communication with the health management server directly based on an Internet-of-Things technology.

In some other embodiments of the present disclosure, data detected by the Internet-of-Things health detection terminal in the health room is collected and transmitted to the health management server based on the Internet-of-Things technology. Illustratively, the health room includes a control terminal and various health detection devices, and the control terminal is configured to control information transmission of the health detection devices.

An operating environment of the Internet-of-Things health detection terminal is open to the public to some extent, so in use, there may exist a risk for the processing and transmission of data. For example, when a detection operation on a previous user has not been completed yet but the device is used by the other user, an abnormal data correspondence occurs. For another example, the data transmission may be adversely affected by such factors as network environment, server response and network attack. Hence, there is a risk for the management and transmission of health data obtained through detection.

Next, an overall process of the health data management method in the embodiments of the present disclosure will be described hereinafter.

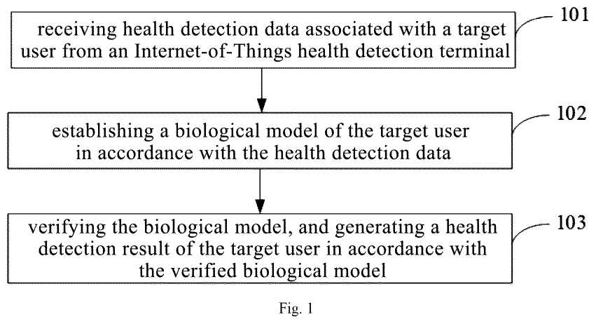

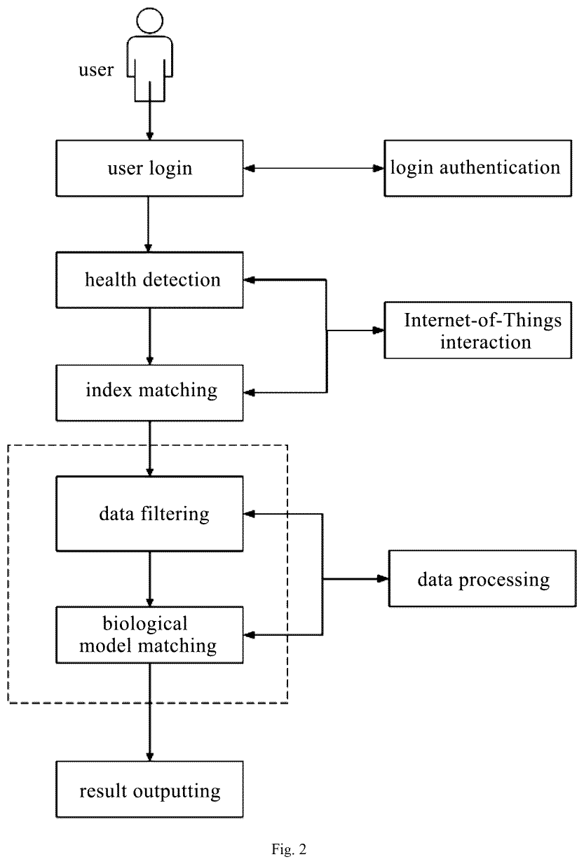

As shown in FIGS. 1 and 2, in some embodiments of the present disclosure, identity information needs to be verified before the Internet-of-Things health detection terminal is used by a user, so as to establish a correspondence between each user and the health detection data.

In a possible embodiment of the present disclosure, login authentication is performed when the user logs into his own account.

In the embodiments of the present disclosure, authentication information for authenticating the login information is stored at the Internet-of-Things health detection terminal, e.g., in a control terminal of the health room. The authentication information may also be stored in the health management server, so as to ensure the verification reliability of the identity information.

In some embodiments of the present disclosure, the verifying the identity information includes: receiving login information from the Internet-of-Things health detection terminal; verifying the login information in accordance with user information about a target user; and transmitting a verification result for the login information to the Internet-of-Things health detection terminal.

During the implementation, the Internet-of-Things health detection terminal collects the login information about the user. In the embodiments of the present disclosure, the user provides the login information in various ways for identity verification. Illustratively, the user inputs a unique account and a corresponding password as the login information, or the user provides the login information through scanning a certificate, performing face recognition, or scanning a code using a mobile phone.

The Internet-of-Things health detection terminal transmits the login information to the health management server. Upon the receipt of the login information, the health management server verifies the identity information in accordance with stored authentication information, and then transmits the verification result to the Internet-of-Things health detection terminal.

After the user's login information has been verified successfully, the user performs health detection using the Internet-of-Things health detection terminal.

Various Internet-of-Things health detection terminals for detecting various health data may be set in the health room.

In a possible embodiment of the present disclosure, various Internet-of-Things health detection terminals are in direct communication with the health management server, and transmit the health detection data to the health management server after obtaining the health detection data.

In another possible embodiment of the present disclosure, each health detection terminal is in communication with a control terminal in different ways, e.g., a wired communication mode (e.g., serial port or data line), an existing or improved data communication network (e.g., Wireless Local Area Network (WLAN), 4^{th}-Generation (4G) mobile network or 4G wireless system, or a 5^{th}-Generation (5G) mobile network or 5G wireless system), or a wireless communication mode (e.g., Bluetooth), which will not be particularly defined herein. The control terminal collects the health detection data obtained by each the Internet-of-Things health detection terminal, and then transmits it to the health management server.

During the implementation, the user is guided to use the Internet-of-Things health detection terminal to perform the health detection through an interaction interface.

As shown in FIG. 1, in a possible embodiment of the present disclosure, the health data management method further includes the following steps.

Step 101: receiving health detection data associated with a target user from the Internet-of-Things health detection terminal.

Upon the receipt of the health detection data from the Internet-of-Things health detection terminal, at first the health management server pre-processes the health management data.

In a possible embodiment of the present disclosure, after the health management server has received the health detection data, the health detection data is matched with preset indices in accordance with a certain rule, and then unified management, normalization and classification processing are performed on the health detection data. Further, the health detection data is classified according to the practical need, so as to form a data set including one or more indices.

In some embodiments of the present disclosure, the pre-processing further includes filtering the health management data so as to reduce abnormal data in the health management data.

In a possible embodiment of the present disclosure, the filtering the health management data includes: invoking a data filtering rule corresponding to the health detection data; and eliminating abnormal data in the health detection data in accordance with the data filtering rule.

In some embodiments of the present disclosure, the data filtering rule includes a numerical range rule. For the numerical range rule, a range of the obtained health detection data is verified, so as to eliminate data not within a biological attribute range.

Illustratively, in the case that the user is healthy or unhealthy, a value of a certain index may fluctuate. To be specific, when the user is healthy, the value of the index is within a normal range, and when the user is unhealthy, the value of the index is within an abnormal range. Both the normal range and the abnormal range belong to the biological attribute range. For a range within which the value of the index never falls no matter whether the user is healthy or unhealthy, when the value of the index falls within such a range, it is considered that the data goes beyond the biological attribute range. At this time, the value of the index may be caused by a detection error or a data transmission error, so it is necessary to delete the data.

For a data collection state rule, a data collection state is verified. For example, when the Internet-of-Things health detection terminal fails to successfully collect data due to a fault, an abnormal value, e.g., a null value, is returned. At this time, it is necessary to filter the abnormal data in accordance with the data collection state.

For a data format rule, a format of the received health detection data is verified so as to determine whether the data format is abnormal. Generally, when the data is normally collected and transmitted, the format thereof meets a certain format rule. When the format is abnormal, it is impossible to perform health analysis subsequently in accordance with the health detection data. In addition, the collection and transmission of the data may also be abnormal, and the data may be inaccurate, so a health analysis result may be adversely affected.

Step 102: establishing a biological model of the target user in accordance with the health detection data.

The biological model refers to a model reflecting a physical state of the user and established in accordance with various health detection data of the user.

In a possible embodiment of the present disclosure, the biological model includes a plurality of detection sub-models corresponding to different detection items, so as to improve analysis accuracy of different health parameters.

In a possible embodiment of the present disclosure, the detection sub-model includes one or more of a blood pressure sub-model, a blood glucose sub-model, a blood oxygen sub-model, a body fat sub-model, a body composition sub-model, a blood pressure sub-model, a bone substance sub-model, a lung function sub-model, an arteriosclerosis sub-model, and an electrocardio sub-model.

Illustratively, the blood pressure sub-model is used to analyze whether a user's blood pressure is abnormal in accordance with various factors such as height, weight, blood pressure, age and gender.

The blood glucose sub-model is used to analyze whether user's blood glucose is normal in accordance with food-intake status, medication habits, height, weight, etc.

The body fat sub-model is used to determine whether a user's body fat content is normal in accordance with height, weight, muscle content, bone mass, body fat content, etc.

During the implementation, monitoring parameters to be collected are determined in accordance with opinions from professionals such as doctors and health coaches, so as to establish the corresponding detection sub-models and improve the professionalism and accuracy of physical state monitoring and analysis. A specific way for establishing each detection sub-model will not be particularly defined herein.

Step 103: verifying the biological model, and generating a health detection result of the target user in accordance with the verified biological model.

It should be appreciated that, the analyzing the biological model includes comparing the user's health detection data with normal data, so as to determine whether the physical state of the user is abnormal in accordance with professional advices or medical knowledge.

The verifying the biological model includes verifying the established biological model so as to determine whether a data error or abnormality is caused due to a detection error or a data transmission error.

In a possible embodiment of the present disclosure, after the health management server has received the health detection data, a data set is established to verify the biological model. In the embodiments of the present disclosure, the data set corresponds to the health detection data of the user. For example, when the health detection data includes a blood pressure-related parameter, it is necessary to establish a data set corresponding to the blood pressure-related parameter, so as to verify the obtained blood pressure-related parameter in accordance with the data set. To be specific, the blood pressure sub-model is verified so as to determine whether a blood pressure-related health state of the user is abnormal.

In some embodiments of the present disclosure, the verifying the biological model includes: verifying a sub-model matching degree of each detection sub-model in accordance with reference data matching the target user; and generating a verification result of the biological model in accordance with the sub-model matching degree.

Taking the blood pressure sub-model as an example, during the implementation, a matching degree of the blood pressure sub-model is verified at first.

During the implementation, at first, a data set corresponding to the blood pressure sub-model, i.e., a blood pressure data set, is obtained, and then a matching degree between data in the blood pressure sub-model and data in the blood pressure data set is obtained.

In a possible embodiment of the present disclosure, the data in the sub-model and the data in the data set of the sub-model are represented as:

{“T”:{
“T1”:[“T1V1”,“T1V2”,...],
“T2”:[“T2V1”,“T2V2”,...],
...
“Tn”:[“TnV1”,“TnV2”,...],
},
“D”:[[“d1”,“d2”,“d3”,...],[“d1”,“d2”,“d3”,...]]},

where T represents a data label of the data in the sub-model data, and D represents a corresponding data set.

In a possible embodiment of the present disclosure, the matching degree of the data is calculated through

$${Si}_{a , b} = \frac{\sum ab - \frac{\sum a \sum b}{n}}{\sqrt{( \sum {a}^{2} - \frac{{( \sum a )}^{2}}{n} ) ( \sum {b}^{2} - \frac{{( \sum b )}^{2}}{n} )}} , \qquad ( 1 )$$

where a represents the data in the blood pressure sub-model, b represents the data for each sample in a blood pressure data set, and n represents the quantity of samples in the blood pressure data set. Hence, the matching degree Si between the data in the blood pressure sub-model and the data in the blood pressure data set is calculated through the above formula (1).

Further, a confidence level Con of the blood pressure sub-model is set by a professional in accordance with experience and professional knowledge.

In some embodiments of the present disclosure, when setting the confidence level Con, a user evaluation mechanism is also introduced. The higher the user evaluation mark, the larger the value of the confidence level Con. In this way, it is able to improve the setting accuracy of the confidence level Con in conjunction with the user's own experience and feelings.

Based on the data matching degree Si of the blood pressure sub-model and the confidence level Con of the blood pressure sub-model, it is able to calculate the sub-model matching degree of the blood pressure sub-model through Mat=Si*Con. The sub-model matching degree Mat of the other detection sub-model may be calculated in a similar way.

Next, a model matching degree of the entire biological model is calculated. With respect to a deviation of the confidence level, a weight p is set for the sub-model based on a grid to which the health room belongs, so as to improve the accuracy of the automatically-calculated confidence level. A value of the weight p is set by a professional in accordance with experience, and optimized and adjusted in accordance with an analysis result.

After determining the sub-model matching degree Mat and the weight p of each detection sub-model, the model matching degree masterMat of the biological model is calculated through masterMat−ΣpₗMatₗ.

When the finally-obtained model matching degree masterMat is greater than a preset matching degree threshold, it is considered that the established biological model is relatively accurate, and there is no abnormality in the data collection, data storage and data transmission. Otherwise, it is considered that there may exist abnormalities in the data collection, data storage and data transmission.

In the case that there are abnormalities in the data collection, data storage and data transmission, the abnormalities are determined through further analysis and verification, and then the data is corrected.

In some other embodiments of the present disclosure, the verifying the sub-model matching degree of each detection sub-model in accordance with the reference data matching the target user includes: obtaining an association relationship among at least a part of the detection sub-models; and verifying the sub-model matching degree of each detection sub-model in accordance with the association relationship.

In some other embodiments of the present disclosure, the sub-model matching degree of the detection sub-model is verified in accordance with not only the matching degree between each detection sub-model and the reference data but also the association relationship among different detection sub-models.

Illustratively, the user's blood pressure is adversely affected by an arteriosclerosis degree of the user to some extent. Hence, the blood pressure sub-model is verified in conjunction with the arteriosclerosis sub-model, so as to improve the accuracy of the verification result for each sub-model.

In some embodiments of the present disclosure, prior to verifying the confidence level of the biological model, the method further includes: obtaining association information about the health detection data, the association information including at least one of an environmental factor or a physical factor of the target user; and generating a constraint condition for verifying the biological model in accordance with the association information.

It should be appreciated that, the health detection data fluctuates to some extent, and a physical state of the user is adversely affected by various environmental factors.

For example, the blood pressure of the user increases within a short time period after exercise; the blood pressure decreases when a temperature increases; and the blood pressure increases when the temperature decreases.

During the implementation, the association information is collected, and the constraint condition for verifying the biological model is adjusted in accordance with the association information, so as to improve the verification accuracy of the biological model.

In some embodiments of the present disclosure, the environmental factors include one or more of a collection period, weather information or a geographical environment; and/or the physical factor includes one or more of food-intake information or health information.

The association information is collected by corresponding sensors in the health room or manually inputted by the user. The collected association information is uploaded to the health management server together with the health detection data, so as to improve the accuracy and reliability of the health detection data.

After the health detection result has been generated, the health detection result is transmitted to the user for reference.

In a possible embodiment of the present disclosure, the health detection. results is transmitted to the health room, e.g., pushed to a control terminal in the health room, so as to be viewed or printed by the user.

In another possible embodiment of the present disclosure, the health detection result is also directly transmitted to a terminal device, e.g., through a mailbox, a specific application or an applet, so as to be viewed by the user.

In a possible embodiment of the present disclosure, in the case of being authorized, a health file is further established to store and maintain the user's health information in accordance with an association relationship between the health detection data and a user's account or identity.

Illustratively, historical health detection results of the user are maintained in the health file for review.

Illustratively, historical data in the health file is viewed in the form of trend charts, lists, individual-item reports or comprehensive reports. The health management server also provides a reminder and proper evaluation with respect to abnormal indices. The user may seeks for body conditioning or medical advices, so as to improve the physical state.

Illustratively, the user may delete, modify or mark the detection data through a client. The health management server automatically performs data synchronization, and processes the data to modify the biological model, so as to create a closed loop for physical sign monitoring and health management.

In a possible embodiment of the present disclosure, the health detection result includes the detected indices, a normal range of each index, abnormal indices, etc., so that the user knows the possible abnormalities. The health detection result further includes an analysis on the abnormalities as well as reasonable suggestions.

In a possible embodiment of the present disclosure, the identity of the user is verified again when pushing the health detection result. When the user passes the verification, the health detection result is pushed as mentioned hereinabove.

When the user fails to pass the verification, a prompt message is sent. To be specific, the prompt message is sent to the user and/or a staff member. Further, a verification result is reviewed, for example, by the staff member, so as to prevent the occurrence of information leakage.

The present disclosure further provides in some embodiments a health data management apparatus applied to a health management server in communication with an Internet-of-Things health detection terminal.

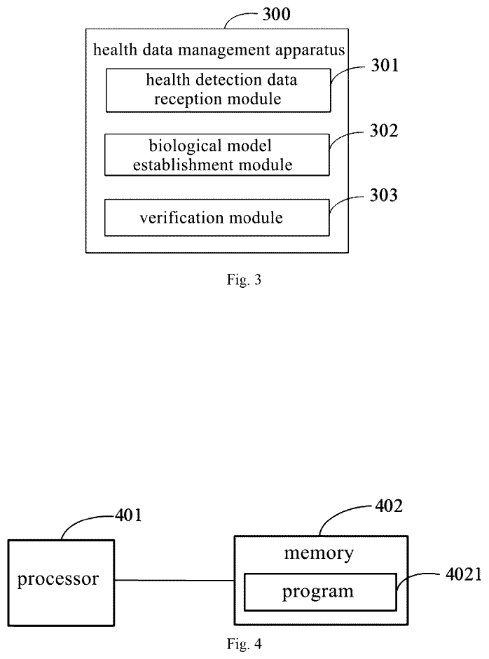

As shown in FIG. 3, in a possible embodiment of the present disclosure, the health data management apparatus includes: a health detection data reception module 301 configured to receive health detection data associated with a target user from the Internet-of-Things health detection terminal; a biological model establishment module 302 configured to establish a biological model of the target user in accordance with the health detection data; and a verification module 303 configured to verify the biological model, and generate a health detection result of the target user in accordance with the verified biological model.

In some embodiments of the present disclosure, the biological model includes a plurality of detection sub-models corresponding to different detection items. The verification module 303 includes: a sub-model matching degree verification sub-module configured to verify a sub-model matching degree of each detection sub-model in accordance with reference data matching the target user, and a verification result generation sub-module configured to generate a verification result of the biological model in accordance with the sub-model matching degree

In some embodiments of the present disclosure, the sub-model matching degree verification sub-module includes: an association relationship obtaining unit configured to obtain an association relationship among at least a part of the detection sub-models; and a verification unit configured to verify the sub-model matching degree of each detection sub-model in accordance with the association relationship.

In some embodiments of the present disclosure, the detection sub-model includes one or more of a blood pressure sub-model, a blood glucose sub-model, a blood oxygen sub-model, a body fat sub-model, a body composition sub-model, a blood pressure sub-model, a bone substance sub-model, a lung function sub-model, an arteriosclerosis sub-model or an electrocardio sub-model.

In some embodiments of the present disclosure, the apparatus further includes: an association information obtaining module configured to obtain association information about the health detection data, the association information including at least one of an environmental factor or a physical factor of the target user; and a constraint condition generation module configured to generate a constraint condition for verifying the biological model in accordance with the association information.

In some embodiments of the present disclosure, the environmental factor includes one or more of a collection period, weather information or geographical environment; and/or the physical factor includes one or more of food-intake information or health information.

In some embodiments of the present disclosure, the apparatus further includes: a data filtering rule invoking module configured to invoke a data filtering rule corresponding to the health detection data, the data filtering rule including one or more of a numerical range rule, a data collection state rule or a data format rule; and a filtration module configured to eliminate abnormal data in the health detection data in accordance with the data filtering rule.

In some embodiments of the present disclosure, the apparatus further includes: a login information reception module configured to receive login information from the Internet-of-Things health detection terminal; a login information verification module configured to verify the login information in accordance with user information about the target user; and a verification result transmission module configured to transmit a verification result for the login information to the Internet-of-Things health detection terminal.

The health data management apparatus 300 in the embodiments of the present disclosure is used to implement the steps of the above-mentioned health data management method with a same technical effect, which will not be particularly defined herein.

The present disclosure further provides in some embodiments an electronic device. Referring to FIG. 4, the electronic device includes a processor 401, a memory 402, and a program 4021 stored in the memory 402 and executed by the processor 401.

The program 4021 is executed by the processor 401 so as to implement the steps of the above-mentioned method with a same technical effect, which will not be particularly defined herein.

It should be appreciated that, all of, or parts of, the steps in the above-mentioned method may be implemented through program-related hardware, and the program may be stored in a readable storage medium.

The present disclosure further provides in some embodiments a readable storage medium storing therein a computer program. The computer program is executed by a processor so as to implement the steps of the above-mentioned method with a same technical effect, which will not be particularly defined herein.

The storage medium includes, for example, Read-Only Memory (ROM), Random Access Memory (RAM), magnetic disk or optical disk.

It should be further appreciated that, the above modules are divided merely on the basis of their logic functions, and in actual use, they may be completely or partially integrated into a physical entity, or physically separated from each other. These modules may be implemented by calling software through a processing element, or implemented in the form of hardware. For example, the determination module may be a processing element arranged separately, or integrated into a chip of the above-mentioned device. In addition, the module may be stored in the memory of the above-mentioned device in the form of a program code, and may be called and executed by a processing element of the above-mentioned device so as to achieve the above functions. The other modules may be implemented in a similar manner. All or parts of the modules may be integrated together or arranged separately. Here, the modules, units or assemblies may each of an Integrated Circuit (IC) having a signal processing capability. During the implementation, the steps of the method or the modules may be implemented through an integrated logic circuit of the processing element in the form of hardware or through instructions in the form of software.

For example, the above modules, units, sub-units or sub-modules may be one or more ICs capable of implementing the above-mentioned method, e.g., one or more Application Specific Integrated Circuits (ASICs), one or more Digital Signal Processors (DSPs), or one or more Field Programmable Gate Array (FPGA). For another example, when a certain module is implemented by calling a program code through a processing element, the processing element may be a general-purpose processor, e.g., a Central Processing Unit (CPU) or any other processor capable of calling the program code. These modules may be integrated together and implemented in the form of system-on-a-chip (SOC).

The above embodiments are for illustrative purposes only, but the present disclosure is not limited thereto. Obviously, a person skilled in the art may make further modifications and improvements without departing from the spirit of the present disclosure, and these modifications and improvements shall also fall within the scope of the present disclosure.

1. A health data management method applied to a health management server in communication with an Internet-of-Things health detection terminal, comprising:
receiving health detection data associated with a target user from the Internet-of-Things health detection terminal;
establishing a biological model of the target user in accordance with the health detection data; and
verifying the biological model, and generating a health detection result of the target user in accordance with the verified biological model.

2. The health data management method according to claim 1, wherein the biological model comprises a plurality of detection sub-models corresponding to different detection items, wherein the verifying the biological model comprises:
verifying a sub-model matching degree of each detection sub-model in accordance with reference data matching the target user; and
generating a verification result of the biological model in accordance with the sub-model matching degree.

3. The health data management method according to claim 2, wherein the verifying the sub-model matching degree of each detection sub-model in accordance with the reference data matching the target user comprises:
obtaining an association relationship among at least a part of the detection sub-models; and
verifying the sub-model matching degree of each detection sub-model in accordance with the association relationship.

4. The health data management method according to claim 2, wherein the detection sub-model comprises one or more of a blood pressure sub-model, a blood glucose sub-model, a blood oxygen sub-model, a body fat sub-model, a body composition sub-model, a bone substance sub-model, a lung function sub-model, an arteriosclerosis sub-model or an electrocardio sub-model.

5. The health data management method according to claim 1, wherein prior to verifying a confidence level of the biological model, the health data management method further comprises:
obtaining association information about the health detection data, the association information comprising at least one of an environmental factor or a physical factor of the target user; and
generating a constraint condition for verifying the biological model in accordance with the association information.

6. The health data management method according to claim 5, wherein the environmental factor comprises one or more of a collection period, weather information or geographical environment; and/or
the physical factor comprises one or more of food-intake information or health information.

7. The health data management method according to claim 1, wherein subsequent to receiving the health detection data associated with the target user from the Internet-of-Things health detection terminal, the health data management method further comprises:
invoking a data filtering rule corresponding to the health detection data, the data filtering rule comprising one or more of a numerical range rule, a data collection state rule or a data format rule; and
eliminating abnormal data in the health detection data in accordance with the data filtering rule.

8. The health data management method according to claim 1, wherein prior to receiving the health detection data associated with the target user from the Internet-of-Things health detection terminal, the health data management method further comprises:
receiving login information from the Internet-of-Things health detection terminal;
verifying the login information in accordance with user information about the target user; and
transmitting a verification result for the login information to the Internet-of-Things health detection terminal.

9. A health data management apparatus applied to a health management server in communication with an Internet-of-Things health detection terminal, comprising:
a health detection data reception module configured to receive health detection data associated with a target user from the Internet-of-Things health detection terminal;
a biological model establishment module configured to establish a biological model of the target user in accordance with the health detection data; and
a verification module configured to verify the biological model, and generate a health detection result of the target user in accordance with the verified biological model.

10. An electronic device, comprising a memory, a processor, and a program stored in the memory and executed by the processor, wherein the processor is configured to read the program in the memory so as to:
receive health detection data associated with a target user from an Internet-of-Things health detection terminal;
establish a biological model of the target user in accordance with the health detection data; and
verify the biological model, and generating a health detection result of the target user in accordance with the verified biological model.

11. A readable storage medium storing therein a program, wherein the program is executed by a processor so as to implement the steps of the health data management method according to claim 1.

12. The health data management method according to claim 3, wherein the detection sub-model comprises one or more of a blood pressure sub-model, a blood glucose sub-model, a blood oxygen sub-model, a body fat sub-model, a body composition sub-model, a bone substance sub-model, a lung function sub-model, an arteriosclerosis sub-model or an electrocardio sub-model.

13. The electronic device according to claim 10, wherein the biological model comprises a plurality of detection sub-models corresponding to different detection items, wherein when verifying the biological model, the processor is specifically configured to:
verify a sub-model matching degree of each detection sub-model in accordance with reference data matching the target user; and
generate a verification result of the biological model in accordance with the sub-model matching degree.

14. The electronic device according to claim 13, wherein when verifying the sub-model matching degree of each detection sub-model in accordance with the reference data matching the target user, the processor is specifically configured to:
obtain an association relationship among at least a part of the detection sub-models; and
verify the sub-model matching degree of each detection sub-model in accordance with the association relationship.

15. The electronic device according to claim 13, wherein the detection sub-model comprises one or more of a blood pressure sub-model, a blood glucose sub-model, a blood oxygen sub-model, a body fat sub-model, a body composition sub-model, a bone substance sub-model, a lung function sub-model, an arteriosclerosis sub-model or an electrocardio sub-model.

16. The electronic device according to claim 14, wherein the detection sub-model comprises one or more of a blood pressure sub-model, a blood glucose sub-model, a blood oxygen sub-model, a body fat sub-model, a body composition sub-model, a bone substance sub-model, a lung function sub-model, an arteriosclerosis sub-model or an electrocardio sub-model.

17. The electronic device according to claim 11, wherein prior to verifying a confidence level of the biological model, the processor is further configured to:
obtain association information about the health detection data, the association information comprising at least one of an environmental factor or a physical factor of the target user; and
generate a constraint condition for verifying the biological model in accordance with the association information.

18. The electronic device according to claim 17, wherein the environmental factor comprises one or more of a collection period, weather information or geographical environment; and/or
the physical factor comprises one or more of food-intake information or health information.

19. The electronic device according to claim 11, wherein subsequent to receiving the health detection data associated with the target user from the Internet-of-Things health detection terminal, the processor is further configured to:
invoke a data filtering rule corresponding to the health detection data, the data filtering rule comprising one or more of a numerical range rule, a data collection state rule or a data format rule; and
eliminate abnormal data in the health detection data in accordance with the data filtering rule.

20. The electronic device according to claim 11, wherein prior to receiving the health detection data associated with the target user from the Internet-of-Things health detection terminal, the processor is further configured to:
receive login information from the Internet-of-Things health detection terminal;
verify the login information in accordance with user information about the target user; and
transmit a verification result for the login information to the Internet-of-Things health detection terminal.
